(19) United States
(12) Patent Application Publication
(10) Pub. No.: US 2015/0152091 A1
(43) Pub. Date: Jun. 4, 2015

(54) CROP ENHANCEMENT

(71) Applicant: Syngenta Participations AG, Basel (CH)

(72) Inventors: Mathilde Denise Lachia, Stein (CH); Alain De Mesmaeker, Stein (CH); Christian Hanno Wolf, Stein (CH); Pierre Joseph Marcel Jung, Stein (CH)

(73) Assignee: Syngenta Participations AG, Basel (CH)

(21) Appl. No.: 14/406,275

(22) PCT Filed: May 28, 2013
(86) PCT No.: PCT/EP2013/060953

§ 371 Date: Dec. 8, 2014

(30) Foreign Application Priority Data
Jun. 11, 2012 (GB) 1210398.2

(51) Int. Cl. C07D 405/12; A01N 43/38

(52) U.S. Cl. CPC C07D 405/12; A01N 43/38

(57) ABSTRACT

The invention relates to a method of crop enhancement. In particular, it relates to a method of enhancing crop plants by applying to the plants, plant parts, plant propagation material, or a plant growing locus, a compound of formula (I), and to the use of said compound for enhancing crops.

The invention relates to a method of crop enhancement. In particular, it relates to a method of enhancing crop plants by applying to the plants, plant parts, plant propagation material, or a plant growing locus, a compound of formula (I), and to the use of said compound for enhancing crops.

Certain methods of enhancing crops are described in the literature. These methods are usually based on conventional fertilising but some also rely on chemicals originally developed for other uses, for example, the insecticide fipronil has been reported e.g. to enhance overall root system and root hair development, increase tiller number and productivity, increase photosynthetic capacity (plant greenness), increase leaf area and plant height and stimulate early flowering and grain maturation and the fungicide pyraclostrobin has been reported to improve plant health e.g. to improve the tolerance to environmental stresses.

International Patent Application Number PCT/EP2011/072303 describes new strigolactam derivatives. It has now been found that these strigolactam derivatives show crop enhancement effects.

Accordingly, the present invention provides a method of enhancing and/or increasing the yield of crop plants by applying to the plants, plant parts, plant propagation material, or a plant growing locus, a compound of formula (I)

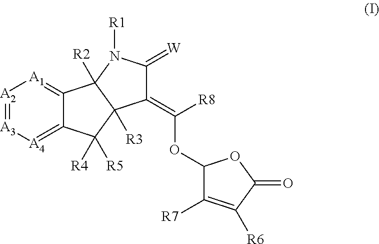

wherein

### W is O or S;

R2 and R3 are independently hydrogen, or C1-C3 alkyl;
R4 and R5 are independently hydrogen, halogen, nitro, cyano, C1-C3 alkyl, C1-C3 haloalkyl, C1-C3 alkoxy, hydroxyl, —OC(O)R9, amine, N— C1-C3 alkyl amine, or N,N-di-C1-C3 alkyl amine;
R9 is hydrogen, C1-C6 alkyl, C1-C6 alkoxy, or C1-C6 haloalkyl;
R6 and R7 are independently hydrogen, C1-C3 alkyl, hydroxyl, or C1-C3 alkoxy;
R8 is hydrogen, nitro, cyano, C1-C6 alkyl, or C1-C6 haloalkyl;
R1 is hydrogen, C1-C6 alkoxy, hydroxyl, amine, N— C1-C6 alkyl amine, N,N-di-C1-C6 alkyl amine, C1-C6 alkyl optionally substituted by one to five R10, C1-C8 alkylcarbonyl, C1-C8 alkoxycarbonyl, aryl optionally substituted by one to five R10, heteroaryl optionally substituted by one to five R10, heterocyclyl optionally substituted by one to five R10, or benzyl optionally substituted by one to five R10;
R10 is hydrogen, cyano, nitro, halogen, C1-C6 alkyl, C1-C6 alkoxy, C1-C6 haloalkyl, C2-C6 alkenyl, or C2-C6 alkynyl;
A₁, A₂, A₃ and A₄ are each independently C—X or nitrogen, wherein each X may be the same or different, and provided that no more than two of A₁, A₂, A₃ and A₄ are nitrogen;
and X is hydrogen, halogen, cyano, hydroxyl, —OC(O)R9, C1-C6 alkoxy, C1-C6 alkyl, C1-C6 haloalkyl, C1-C3 hydroxyalkyl, nitro, amine, N— C1-C6 alkyl amine, N,N-di-C1-C6 alkyl amine, or NHC(O)R9;
or a composition comprising said compound.

In preferred compounds of formula (I), independently from each other,

### W is O;

R2 and R3 are independently hydrogen, methyl or ethyl;
R4 and R5 are independently hydrogen, hydroxyl, methyl or ethyl;
R6, R7 and R8 are independently hydrogen, methyl or ethyl;
R1 is hydrogen, C1-C6 alkoxy, C1-C6 alkyl optionally substituted by one to five R10, C1-C8 alkylcarbonyl, C1-C8 alkoxycarbonyl, aryl optionally substituted by one to five R10, heteroaryl optionally substituted by one to five R10, heterocyclyl optionally substituted by one to five R10, or benzyl optionally substituted by one to five R10;
R10 is independently hydrogen, cyano, nitro, halogen, C1-C6 alkyl, C1-C6 alkoxy or C1-C6 haloalkyl;
A₁, A₂, A₃ and A₄ are each independently C-X; and
X is hydrogen, hydroxyl, halogen, cyano, methyl, ethyl, n-propyl, hydroxymethyl, trifluoromethyl or methoxy.

Especially preferred compounds of formula (I) are those, wherein

W is oxygen;
R2, R3, R7 and R8 are hydrogen;
R4 and R5 are independently hydrogen or hydroxyl;
R6 is methyl;
R1 is hydrogen, C1-C6 alkoxy, C1-C6 alkyl optionally substituted by one to five R10, C1-C8 alkylcarbonyl, C1-C8 alkoxycarbonyl, benzyl optionally substituted by one to five R10;
R10 is hydrogen, cyano, nitro, chloride, bromine, fluorine, methyl, methoxy and trifluoromethyl;

### A₁, A₂, A₃ and A₄ are C-X; and

X is hydrogen, hydroxyl, methyl, trifluoromethyl or methoxy.

Further compounds of formula (I) are preferred, wherein R1 is selected from the group consisting of hydrogen, methyl, ethyl, phenyl, benzyl, acetate, and methoxycarbonyl.

Further compounds of formula (I) are preferred, wherein X is selected from the group consisting of hydrogen, methyl, hydroxyl and methoxy.

Methods of making such compounds are disclosed in International Patent Application Number PCT/EP2011/072303.

The term “increasing the yield” of a plant means that the yield of a product of the plant is increased by a measurable amount over the yield of the same product of the plant produced under the same conditions, but without the application of the combinations according to the present invention. It is preferred that the yield is increased by at least about 0.5%, preferably 1%, more preferably 2%, yet more preferably 4% or more. Even more preferred is an increase in yield of at least about 5%, 10%, 15% or 20% or more.

According to the present invention, ‘crop enhancement’ means an improvement in plant vigour, an improvement in plant quality, improved tolerance to stress factors, and/or improved input use efficiency.

According to the present invention, an ‘improvement in plant vigour’ means that certain traits are improved qualitatively or quantitatively when compared with the same trait in a control plant which has been grown under the same conditions in the absence of the method of the invention. Such traits include, but are not limited to, early and/or improved germination, improved emergence, the ability to use less seeds, increased root growth, a more developed root system, increased root nodulation, increased shoot growth, increased tillering, stronger tillers, more productive tillers, increased or improved plant stand, less plant verse (lodging), an increase and/or improvement in plant height, an increase in plant weight (fresh or dry), bigger leaf blades, greener leaf colour, increased pigment content, increased photosynthetic activity, earlier flowering, longer panicles, early grain maturity, increased seed, fruit or pod size, increased pod or ear number, increased seed number per pod or ear, increased seed mass, enhanced seed filling, less dead basal leaves, delay of senescence, improved vitality of the plant, increased levels of amino acids in storage tissues and/or less inputs needed (e.g. less fertiliser, water and/or labour needed). A plant with improved vigour may have an increase in any of the aforementioned traits or any combination or two or more of the aforementioned traits.

According to the present invention, an ‘improvement in plant quality’ means that certain traits are improved qualitatively or quantitatively when compared with the same trait in a control plant which has been grown under the same conditions in the absence of the method of the invention. Such traits include, but are not limited to, improved visual appearance of the plant, reduced ethylene (reduced production and/or inhibition of reception), improved quality of harvested material, e.g. seeds, fruits, leaves, vegetables (such improved quality may manifest as improved visual appearance of the harvested material), improved carbohydrate content (e.g. increased quantities of sugar and/or starch, improved sugar acid ratio, reduction of reducing sugars, increased rate of development of sugar), improved protein content, improved oil content and composition, improved nutritional value, reduction in anti-nutritional compounds, improved organoleptic properties (e.g. improved taste) and/or improved consumer health benefits (e.g. increased levels of vitamins and anti-oxidants)), improved post-harvest characteristics (e.g. enhanced shelf-life and/or storage stability, easier processability, easier extraction of compounds), more homogenous crop development (e.g. synchronised germination, flowering and/or fruiting of plants), and/or improved seed quality (e.g. for use in following seasons). A plant with improved quality may have an increase in any of the aforementioned traits or any combination or two or more of the aforementioned traits.

According to the present invention, an ‘improved tolerance to stress factors’ means that certain traits are improved qualitatively or quantitatively when compared with the same trait in a control plant which has been grown under the same conditions in the absence of the method of the invention. Such traits include, but are not limited to, an increased tolerance and/or resistance to abiotic stress factors which cause sub-optimal growing conditions such as drought (e.g. any stress which leads to a lack of water content in plants, a lack of water uptake potential or a reduction in the water supply to plants), cold exposure, heat exposure, osmotic stress, UV stress, flooding, increased salinity (e.g. in the soil), increased mineral exposure, ozone exposure, high light exposure and/or limited availability of nutrients (e.g. nitrogen and/or phosphorus nutrients). A plant with improved tolerance to stress factors may have an increase in any of the aforementioned traits or any combination or two or more of the aforementioned traits. In the case of drought and nutrient stress, such improved tolerances may be due to, for example, more efficient uptake, use or retention of water and nutrients.

According to the present invention, an ‘improved input use efficiency’ means that the plants are able to grow more effectively using given levels of inputs compared to the grown of control plants which are grown under the same conditions in the absence of the method of the invention. In particular, the inputs include, but are not limited to fertiliser (such as nitrogen, phosphorous, potassium, micronutrients), light and water. A plant with improved input use efficiency may have an improved use of any of the aforementioned inputs or any combination of two or more of the aforementioned inputs.

Other crop enhancements of the present invention include a decrease in plant height, or reduction in tillering, which are beneficial features in crops or conditions where it is desirable to have less biomass and fewer tillers.

Crop enhancement also includes safening of crop plants against phytotoxic effects of pesticides or other compounds that are applied to the crop.

Any or all of the above crop enhancements may lead to an improved yield by improving e.g. plant physiology, plant growth and development and/or plant architecture. In the context of the present invention ‘yield’ includes, but is not limited to, (i) an increase in biomass production, grain yield, starch content, oil content and/or protein content, which may result from (a) an increase in the amount produced by the plant per se or (b) an improved ability to harvest plant matter, (ii) an improvement in the composition of the harvested material (e.g. improved sugar acid ratios, improved oil composition, increased nutritional value, reduction of anti-nutritional compounds, increased consumer health benefits) and/or (iii) an increased/facilitated ability to harvest the crop, improved processability of the crop and/or better storage stability/shelf life. Increased yield of an agricultural plant means that, where it is possible to take a quantitative measurement, the yield of a product of the respective plant is increased by a measurable amount over the yield of the same product of the plant produced under the same conditions, but without application of the present invention. According to the present invention, it is preferred that the yield be increased by at least 0.5%, more preferred at least 1%, even more preferred at least 2%, still more preferred at least 4%, preferably 5% or even more.

Any or all of the above crop enhancements may also lead to an improved utilisation of land, i.e. land which was previously unavailable or sub-optimal for cultivation may become available. For example, plants which show an increased ability to survive in drought conditions, may be able to be cultivated in areas of sub-optimal rainfall, e.g. perhaps on the fringe of a desert or even the desert itself.

In one aspect of the present invention, crop enhancements are made in the substantial absence of pressure from pests and/or diseases and/or abiotic stress. In a further aspect of the present invention, improvements in plant vigour, stress tolerance, quality and/or yield are made in the substantial absence of pressure from pests and/or diseases. For example pests and/or diseases may be controlled by a pesticidal treatment that is applied prior to, or at the same time as, the method of the present invention. In a still further aspect of the present invention, improvements in plant vigour, stress tolerance, quality and/or yield are made in the absence of pest and/or disease pressure. In a further embodiment, improvements in plant vigour, quality and/or yield are made in the absence, or substantial absence, of abiotic stress.

According to the present invention, there is provided the use of a compound of formula (I) or a composition comprising a compound of formula (I) for improving plant yield, plant vigour, plant quality, plant tolerance to stress factors and/or plant input use efficiency.

In one aspect of the present invention, improvements in plant vigour, stress tolerance, quality, yield and/or input use efficiency are made in the substantial absence of pressure from pests and/or diseases. For example pests and/or diseases may be controlled by a pesticidal treatment that is applied prior to, or at the same time as, the method of the present invention.

In a still further aspect of the present invention, improvements in plant vigour, stress tolerance, quality, yield and/or input use efficiency are made in the absence of pest and/or disease pressure. In a further embodiment, improvements in plant vigour, quality, yield and/or input use efficiency are made in the absence, or substantial absence, of abiotic stress.

In one aspect of the invention, there is provided a method for improving plant yield, comprising applying to a plant, plant part, plant propagation material, or a plant growing locus, a compound of formula (I).

In one aspect of the invention, there is provided a method for improving plant input use efficiency, comprising applying to a plant, plant part, plant propagation material, or a plant growing locus, a compound of formula (I).

In one aspect of the invention, there is provided a method for improving plant vigour and/or plant quality, and/or plant tolerance to stress factors, comprising applying to a plant, plant part, plant propagation material, or a plant growing locus, a compound of formula (I).

Suitably, the plant has improved tolerance to drought conditions.

Suitably the plant exhibits a higher nitrogen balance index, increased root surface area, and/or increase number of open flowers compared to untreated plants.

Suitably the compound or composition is present in an effective amount. More suitably, the compound or composition is present in an amount effective to enhance the plant.

Crop enhancement may be achieved in a range of crops. Suitable target crops are, in particular, cereals, such as wheat, barley, rye, oats, rice, maize or sorghum; beet, such as sugar or fodder beet; fruit, for example pomaceous fruit, stone fruit or soft fruit, such as apples, pears, plums, peaches, almonds, cherries or berries, for example strawberries, raspberries or blackberries; leguminous crops, such as beans, lentils, peas or soybean; oil crops, such as oilseed rape, mustard, poppies, olives, sunflowers, coconut, castor, cocoa or ground nuts; cucurbits, such as pumpkins, cucumbers or melons; fibre plants, such as cotton, flax, hemp or jute; citrus fruit, such as oranges, lemons, grapefruit or tangerines; vegetables, such as spinach, lettuce, asparagus, cabbages, carrots, onions, tomatoes, potatoes or bell peppers; Lauraceae, such as avocado, Cinnamonium or camphor; and also tobacco, nuts, coffee, eggplants, sugarcane, tea, pepper, grapevines, hops, the plantain family, latex plants, turfgrass (such as warm season and cool season turf) and ornamentals (such as bedding plants, flowering plants, shrubs, and trees). Preferably the crop plants are selected from the group consisting of corn, wheat, rice, soybean.

The term “crops” is to be understood as including also crops that have been modified as a result of conventional methods of breeding, or via genetic engineering, to impart desirable traits such as tolerance to herbicides, resistance to insects or disease, tolerance to abiotic stress such as drought, heat or salt, or enhanced yield or quality.

For example the term “crops” includes plants that have been rendered tolerant to herbicides like bromoxynil or classes of herbicides (such as, for example, HPPD inhibitors, ALS inhibitors, for example primisulfuron, prosulfuron and trifloxysulfuron, EPSPS (5-enol-pyrovyl-shikimate-3-phosphate-synthase) inhibitors, GS (glutamine synthetase) inhibitors). An example of a crop that has been rendered tolerant to imidazolinones, e.g. imazamox, by conventional methods of breeding (mutagenesis) is Clearfield® summer rape (Canola). Examples of crops that have been rendered tolerant to herbicides or classes of herbicides by genetic engineering methods include glyphosate- and glufosinate-resistant maize varieties commercially available under the trade names RoundupReady® and LibertyLink®.

The term “crops” is also to be understood as including also crop plants which have been so transformed by the use of recombinant DNA techniques that they are capable of synthesising one or more selectively acting toxins, such as are known, for example, from toxin-producing bacteria, especially those of the genus Bacillus.

Toxins that can be expressed by such transgenic plants include, for example, insecticidal proteins, for example insecticidal proteins from Bacillus cereus or Bacillus popliae; or insecticidal proteins from Bacillus thuringiensis, such as δ-endotoxins, e.g. CryIA(b), CryIA(c), CryIF, CryIF(a2), CryIIA(b), CryIIIA, CryIIIB(b1) or Cry9c, or vegetative insecticidal proteins (VIP), e.g. VIP1, VIP2, VIP3 or VIP3A; or insecticidal proteins of bacteria colonising nematodes, for example Photorhabdus spp. or Xenorhabdus spp., such as Photorhabdus luminescens, Xenorhabdus nematophilus; toxins produced by animals, such as scorpion toxins, arachnid toxins, wasp toxins and other insect-specific neurotoxins; toxins produced by fungi, such as Streptomycetes toxins, plant lectins, such as pea lectins, barley lectins or snowdrop lectins; agglutinins; proteinase inhibitors, such as trypsine inhibitors, serine protease inhibitors, patatin, cystatin, papain inhibitors; ribosome-inactivating proteins (RIP), such as ricin, maize-RIP, abrin, luffin, saporin or bryodin; steroid metabolism enzymes, such as 3-hydroxysteroidoxidase, ecdysteroid-UDP-glycosyl-transferase, cholesterol oxidases, ecdysone inhibitors, HMG-COA-reductase, ion channel blockers, such as blockers of sodium or calcium channels, juvenile hormone esterase, diuretic hormone receptors, stilbene synthase, bibenzyl synthase, chitinases and glucanases. The term “crops” is to be understood as including also crop plants which have been so transformed by the use of recombinant DNA techniques that they are capable of synthesising antipathogenic substances having a selective action, such as, for example, the so-called “pathogenesis-related proteins” (PRPs, see e.g. EP-A-0 392 225). Examples of such antipathogenic substances and transgenic plants capable of synthesising such antipathogenic substances are known, for example, from EP-A-0 392 225, WO 95/33818, and EP-A-0 353 191. The methods of producing such transgenic plants are generally known to the person skilled in the art and are described, for example, in the publications mentioned above.

Antipathogenic substances which can be expressed by such transgenic plants include, for example, ion channel blockers, such as blockers for sodium and calcium channels, for example the viral KP1, KP4 or KP6 toxins; stilbene synthases; bibenzyl synthases; chitinases; glucanases; the so-called “pathogenesis-related proteins” (PRPs; see e.g. EP-A-0 392 225); antipathogenic substances produced by microorganisms, for example peptide antibiotics or heterocyclic antibiotics (see e.g. WO 95/33818) or protein or polypeptide factors involved in plant pathogen defence (so-called “plant disease resistance genes”, as described in WO 03/000906).

Crops may also be modified for enhanced resistance to fungal (for example Fusarium, Anthracnose, or Phytophthora), bacterial (for example Pseudomonas) or viral (for example potato leafroll virus, tomato spotted wilt virus, cucumber mosaic virus) pathogens.

Crops also include those that have enhanced resistance to nematodes, such as the soybean cyst nematode.

Crops that are tolerance to abiotic stress include those that have enhanced tolerance to drought, high salt, high temperature, chill, frost, or light radiation, for example through expression of NF-YB or other proteins known in the art.

Crops that exhibit enhanced yield or quality include those with improved flowering or fruit ripening properties (such as delayed ripening); modified oil, starch, amino acid, fatty acid, vitamin, phenolic or other content (such as Vistive™ soybean variety); enhanced nutrient utilisation (such as improved nitrogen assimilation); and enhanced quality plant product (such as higher quality cotton fibre).

In one aspect of the present invention, the compound of formula (I) is applied in the form of a composition, further comprising an agriculturally acceptable carrier.

The compounds of formula (I) are generally applied as compositions such as emulsifiable concentrates, suspension concentrates, directly sprayable or dilutable solutions, spreadable pastes, dilute emulsions, soluble powders, dispersible powders, wettable powders, dusts, granules or encapsulations in polymeric substances, which comprise—at least—one of the active ingredients according to the invention and which are to be selected to suit the intended aims and the prevailing circumstances.

In these compositions, the active ingredient is employed in pure form, a solid active ingredient for example in a specific particle size, or, preferably, together with—at least—one of the auxiliaries conventionally used in the art of formulation, such as extenders, for example solvents or solid carriers, or such as surface-active compounds (surfactants).

Examples of suitable solvents are: unhydrogenated or partially hydrogenated aromatic hydrocarbons, preferably the fractions C8 to C12 of alkylbenzenes, such as xylene mixtures, alkylated naphthalenes or tetrahydronaphthalene, aliphatic or cycloaliphatic hydrocarbons, such as paraffins or cyclohexane, alcohols such as ethanol, propanol or butanol, glycols and their ethers and esters such as propylene glycol, dipropylene glycol ether, ethylene glycol or ethylene glycol monomethyl ether or ethylene glycol monoethyl ether, ketones, such as cyclohexanone, isophorone or diacetone alcohol, strongly polar solvents, such as N-methylpyrrolid-2-one, dimethyl sulfoxide or N,N-dimethylformamide, water, unepoxidized or epoxidized vegetable oils, such as unexpodized or epoxidized rapeseed, castor, coconut or soya oil, and silicone oils.

Solid carriers which are used for example for dusts and dispersible powders are, as a rule, ground natural minerals such as calcite, talc, kaolin, montmorillonite or attapulgite. To improve the physical properties, it is also possible to add highly disperse silicas or highly disperse absorbtive polymers. Suitable particulate adsorptive carriers for granules are porous types, such as pumice, brick grit, sepiolite or bentonite, and suitable non-sorptive carrier materials are calcite or sand. In addition, a large number of granulated materials of inorganic or organic nature can be used, in particular dolomite or comminuted plant residues.

Suitable surface-active compounds are, depending on the type of the active ingredient to be formulated, non-ionic, cationic and/or anionic surfactants or surfactant mixtures which have good emulsifying, dispersing and wetting properties. The surfactants mentioned below are only to be considered as examples; a large number of further surfactants which are conventionally used in the art of formulation and suitable according to the invention are described in the relevant literature.

Suitable non-ionic surfactants are, especially, polyglycol ether derivatives of aliphatic or cycloaliphatic alcohols, of saturated or unsaturated fatty acids or of alkyl phenols which may contain approximately 3 to approximately 30 glycol ether groups and approximately 8 to approximately 20 carbon atoms in the (cyclo)aliphatic hydrocarbon radical or approximately 6 to approximately 18 carbon atoms in the alkyl moiety of the alkyl phenols. Also suitable are water-soluble polyethylene oxide adducts with polypropylene glycol, ethylenediaminopo [symbol] lypropylene glycol or alkyl polypropylene glycol having 1 to approximately 10 carbon atoms in the alkyl chain and approximately 20 to approximately 250 ethylene glycol ether groups and approximately 10 to approximately 100 propylene glycol ether groups. Normally, the abovementioned compounds contain 1 to approximately 5 ethylene glycol units per propylene glycol unit. Examples which may be mentioned are nonylphenoxypolyethoxyethanol, castor oil polyglycol ether, polypropylene glycol/polyethylene oxide adducts, tributylpheno [symbol] xypolyethoxyethanol, polyethylene glycol or octylphenoxypolyethoxyethanol. Also suitable are fatty acid esters of polyoxyethylene sorbitan, such as polyoxyethylene sorbitan trioleate.

The cationic surfactants are, especially, quarternary ammonium salts which generally have at least one alkyl radical of approximately 8 to approximately 22 C atoms as substituents and as further substituents (unhalogenated or halogenated) lower alkyl or hydroxyalkyl or benzyl radicals. The salts are preferably in the form of halides, methylsulfates or ethylsulfates. Examples are stearyltrimethylammonium chloride and benzylbis(2-chloroethyl)ethyl [symbol] ammonium bromide.

Examples of suitable anionic surfactants are water-soluble soaps or water-soluble synthetic surface-active compounds. Examples of suitable soaps are the alkali, alkaline earth or (unsubstituted or substituted) ammonium salts of fatty acids having approximately 10 to approximately 22 C atoms, such as the sodium or potassium salts of oleic or stearic acid, or of natural fatty acid mixtures which are obtainable for example from coconut or tall oil; mention must also be made of the fatty acid methyl taurates. However, synthetic surfactants are used more frequently, in particular fatty sulfonates, fatty sulfates, sulfonated benzimidazole derivatives or alkylaryl sulfonates. As a rule, the fatty sulfonates and fatty sulfates are present as alkali, alkaline earth or (substituted or unsubstituted) ammonium salts and they generally have an alkyl radical of approximately 8 to approximately 22 C atoms, alkyl also to be understood as including the alkyl moiety of acyl radicals; examples which may be mentioned are the sodium or calcium salts of lignosulfonic acid, of the dodecylsulfuric ester or of a fatty alcohol sulfate mixture prepared from natural fatty acids. This group also includes the salts of the sulfuric esters and sulfonic acids of fatty alcohol/ethylene oxide adducts. The sulfonated benzimidazole derivatives preferably contain 2 sulfonyl groups and a fatty acid radical of approximately 8 to approximately 22 C atoms. Examples of alkylarylsulfonates are the sodium, calcium or triethanolammonium salts of decylbenzenesulfonic acid, of dibutylnaphthalenesulfonic acid or of a naphthalenesulfonic acid/formaldehyde condensate. Also possible are, furthermore, suitable phosphates, such as salts of the phosphoric ester of a p-nonylphenol/(4-14)ethylene oxide adduct, or phospho lipids. Further suitable phosphates are tris-esters of phosphoric acid with aliphatic or aromatic alcohols and/or bis-esters of alkyl phosphonic acids with aliphatic or aromatic alcohols, which are a high performance oil-type adjuvant. These tris-esters have been described, for example, in WO0147356, WO0056146, EP-A-0579052 or EP-A-1018299 or are commercially available under their chemical name. Preferred tris-esters of phosphoric acid for use in the new compositions are tris-(2-ethylhexyl)phosphate, tris-n-octyl phosphate and tris-butoxyethyl phosphate, where tris-(2-ethylhexyl)phosphate is most preferred. Suitable bis-ester of alkyl phosphonic acids are bis-(2-ethylhexyl)-(2-ethylhexyl)-phosphonate, bis-(2-ethylhexyl)-(n-octyl)-phosphonate, dibutyl-butyl phosphonate and bis(2-ethylhexyl)-tripropylene-phosphonate, where bis-(2-ethylhexyl)-(n-octyl)-phosphonate is particularly preferred.

The compositions according to the invention can preferably additionally include an additive comprising an oil of vegetable or animal origin, a mineral oil, alkyl esters of such oils or mixtures of such oils and oil derivatives. The amount of oil additive used in the composition according to the invention is generally from 0.01 to 10%, based on the spray mixture. For example, the oil additive can be added to the spray tank in the desired concentration after the spray mixture has been prepared. Preferred oil additives comprise mineral oils or an oil of vegetable origin, for example rapeseed oil such as ADIGOR® and MERO®, olive oil or sunflower oil, emulsified vegetable oil, such as AMIGO® (Rhone-Poulenc Canada Inc.), alkyl esters of oils of vegetable origin, for example the methyl derivatives, or an oil of animal origin, such as fish oil or beef tallow. A preferred additive contains, for example, as active components essentially 80% by weight alkyl esters of fish oils and 15% by weight methylated rapeseed oil, and also 5% by weight of customary emulsifiers and pH modifiers. Especially preferred oil additives comprise alkyl esters of C₈-C₂₂ fatty acids, especially the methyl derivatives of C₁₂-C₁₈ fatty acids, for example the methyl esters of lauric acid, palmitic acid and oleic acid, being important. Those esters are known as methyl laurate (CAS-111-82-0), methyl palmitate (CAS-112-39-0) and methyl oleate (CAS-112-62-9). A preferred fatty acid methyl ester derivative is Emery® 2230 and 2231 (Cognis GmbH). Those and other oil derivatives are also known from the Compendium of Herbicide Adjuvants, 5th Edition, Southern Illinois University, 2000. Also, alkoxylated fatty acids can be used as additives in the inventive compositions as well as polymethylsiloxane based additives, which have been described in WO08/037373.

The application and action of the oil additives can be further improved by combining them with surface-active substances, such as non-ionic, anionic or cationic surfactants. Examples of suitable anionic, non-ionic and cationic surfactants are listed on pages 7 and 8 of WO 97/34485. Preferred surface-active substances are anionic surfactants of the dodecyl-benzylsulfonate type, especially the calcium salts thereof, and also non-ionic surfactants of the fatty alcohol ethoxylate type. Special preference is given to ethoxylated C₁₂-C₂₂ fatty alcohols having a degree of ethoxylation of from 5 to 40. Examples of commercially available surfactants are the Genapol types (Clariant AG). Also preferred are silicone surfactants, especially polyalkyl-oxide-modified heptamethyltrisiloxanes, which are commercially available e.g. as Silwet L-77®, and also perfluorinated surfactants. The concentration of surface-active substances in relation to the total additive is generally from 1 to 30% by weight. Examples of oil additives that consist of mixtures of oils or mineral oils or derivatives thereof with surfactants are Edenor ME SU®, Turbocharge® (Syngenta AG, CH) and Actipron® (BP Oil UK Limited, GB).

The said surface-active substances may also be used in the formulations alone, that is to say without oil additives.

Furthermore, the addition of an organic solvent to the oil additive/surfactant mixture can contribute to a further enhancement of action. Suitable solvents are, for example, Solvesso® (ESSO) and Aromatic Solvent® (Exxon Corporation). The concentration of such solvents can be from 10 to 80% by weight of the total weight. Such oil additives, which may be in admixture with solvents, are described, for example, in U.S. Pat. No. 4,834,908. A commercially available oil additive disclosed therein is known by the name MERGE® (BASF Corporation). A further oil additive that is preferred according to the invention is SCORE® (Syngenta Crop Protection Canada.)

In addition to the oil additives listed above, in order to enhance the activity of the compositions according to the invention it is also possible for formulations of alkylpyrrolidones, (e.g. Agrimax®) to be added to the spray mixture. Formulations of synthetic latices, such as, for example, polyacrylamide, polyvinyl compounds or poly-1-p-menthene (e.g. Bond®, Courier® or Emerald®) can also be used. Solutions that contain propionic acid, for example Eurogkem Pen-e-trate®, can also be mixed into the spray mixture as activity-enhancing agents.

As a rule, the compositions comprise from 0.1 to 99%, especially from 0.1 to 95%, of active ingredient of the compound of formula (I). The compositions generally comprise from 1 to 99.9%, especially from 5 to 99.9%, of at least one solid or liquid adjuvant, it being possible as a rule for 0 to 25%, especially 0.1 to 20%, of the composition to be surfactants (% in each case meaning percent by weight). Whereas concentrated compositions tend to be preferred for commercial goods, the end consumer as a rule uses dilute compositions which have substantially lower concentrations of active ingredient.

The compound of formula (I) is applied to the plant, plant locus or plant propagation material at a rate from 0.1 to 200 g ai/ha, suitably from 0.5 to 100 g ai/ha, preferably from 1 to 100 g ai/ha.

The compositions can also comprise further solid or liquid auxiliaries, such as stabilizers, for example unepoxidized or epoxidized vegetable oils (for example epoxidized coconut oil, rapeseed oil or soya oil), antifoams, for example silicone oil, preservatives, viscosity regulators, binders and/or tackifiers; fertilizers, in particular nitrogen containing fertilizers such as ammonium nitrates and urea as described in WO08/017388, which can enhance the efficacy of the inventive compounds; or other active ingredients for achieving specific effects, for example ammonium or phosphonium salts, in particular halides, (hydrogen)sulphates, nitrates, (hydrogen)carbonates, citrates, tartrates, formiates and acetates, as described in WO07/068427 and WO07/068428, which also can enhance the efficacy of the inventive compounds and which can be used in combination with penetration enhancers such as alkoxalated fatty acids; bactericides, fungicides, nematocides, plant activators, molluscicides or herbicides.

The compositions used according to the invention are prepared in a manner known per se, in the absence of auxiliaries for example by grinding, screening and/or compressing a solid active ingredient and in the presence of at least one auxiliary for example by intimately mixing and/or grinding the active ingredient with the auxiliary (auxiliaries). These processes for the preparation of the compositions and the use of the compounds I for the preparation of these compositions are also a subject of the invention.

The application methods for the compositions, that is the methods of enhancing crops, such as spraying, atomizing, dusting, brushing on, dressing, scattering or pouring—which are to be selected to suit the intended aims of the prevailing circumstances—and the use of the compositions for enhancing crops of the abovementioned type are other subjects of the invention. Typical rates of concentration are between 0.1 and 1000 ppm, preferably between 0.1 and 500 ppm, of active ingredient. The rate of application per hectare is generally 1 to 2000 g of active ingredient per hectare, in particular 10 to 1000 g/ha, preferably 10 to 600 g/ha. In particular, for soil application on field crops, the rates is preferably 10 to 150 g/ha, and for soil application on vegetables, the rates is preferably 5 to 100 g/ha. For foliar application on field crops, preferably 50 to 200 g/ha are used.

One preferred method of application in the field of crop protection is application to the foliage of the plants (foliar application), it being possible to select frequency and rate of application to match the danger of infestation with the pest in question. Alternatively, the active ingredient can reach the plants via the root system (systemic action), by drenching the locus of the plants with a liquid composition or by incorporating the active ingredient in solid form into the locus of the plants, for example into the soil, for example in the form of granules (for soil application, or for surface broadcast). In the case of paddy rice crops, such granules can be metered into the flooded paddy-field.

The compositions used according to the invention are also suitable for the protection of plant propagation material, for example seeds, such as fruit, tubers or kernels, or nursery plants, against pests of the abovementioned type. The propagation material can be treated with the compositions prior to planting, for example seed can be treated prior to sowing.

Alternatively, the compositions can be applied to seed kernels (coating), either by soaking the kernels in a liquid composition or by applying a layer of a solid composition. It is also possible to apply the compositions when the propagation material is planted to the site of application, for example into the seed furrow during drilling. These treatment methods for plant propagation material and the plant propagation material thus treated are further subjects of the invention.

Although it is believed that the present method can be applied to a seed in any physiological state, it is preferred that the seed be in a sufficiently durable state that it incurs no damage during the treatment process. Typically, the seed would be a seed that had been harvested from the field; removed from the plant; and separated from any cob, stalk, outer husk, and surrounding pulp or other non-seed plant material. The seed would preferably also be biologically stable to the extent that the treatment would cause no biological damage to the seed. It is believed that the treatment can be applied to the seed at any time between harvest of the seed and sowing of the seed or during the sowing process (seed directed applications). The seed may also be primed either before or after the treatment.

Even distribution of the compound and adherence thereof to the seeds is desired during propagation material treatment. Treatment could vary from a thin film (dressing) of a formulation containing the compound, for example, a mixture of active ingredient(s), on a plant propagation material, such as a seed, where the original size and/or shape are recognizable to an intermediary state (such as a coating) and then to a thicker film (such as pelleting with many layers of different materials (such as carriers, for example, clays; different formulations, such as of other active ingredients; polymers; and colourants) where the original shape and/or size of the seed is no longer recognisable into the controlled release material or applied between layers of materials, or both.

The seed treatment occurs to an unsown seed, and the term “unsown seed” is meant to include seed at any period between the harvest of the seed and the sowing of the seed in the ground for the purpose of germination and growth of the plant.

Treatment to an unsown seed is not meant to include those practices in which the active ingredient is applied to the soil but would include any application practice that would target the seed during the planting process.

Preferably, the treatment occurs before sowing of the seed so that the sown seed has been pre-treated with the compound. In particular, seed coating or seed pelleting are preferred in the treatment of the compound. As a result of the treatment, the compound is adhered on to the seed and therefore available for pest control.

The treated seeds can be stored, handled, sowed and tilled in the same manner as any other active ingredient treated seed.

Further methods of application of the compositions used according to the invention comprise drip application onto the soil, dipping of parts of plants such as roots bulbs or tubers, drenching the soil, as well as soil injection. These methods are known in the art.

In order to apply a compound of formula (I) for enhancing crops, a compound of formula (I) is usually formulated into a composition which includes, in addition to the compound of formula (I), a suitable inert diluent or carrier and, optionally, a formulation adjuvant in form of a surface active agent (SFA) as described herein or, for example, in EP-B-1062217. SFAs are chemicals which are able to modify the properties of an interface (for example, liquid/solid, liquid/air or liquid/liquid interfaces) by lowering the interfacial tension and thereby leading to changes in other properties (for example dispersion, emulsification and wetting). It is preferred that all compositions (both solid and liquid formulations) comprise, by weight, 0.0001 to 95%, more preferably 1 to 85%, for example 5 to 60%, of a compound of formula (I). The composition is generally used for the control of pests such that a compound of formula (I) is applied at a rate of from 0.1 g to 10 kg per hectare, preferably from 1 g to 6 kg per hectare, more preferably from 1 g to 1 kg per hectare, even more preferably from 25 g to 200 g per hectare, and particularly from 50 g to 100 g per hectare.

When used in a seed dressing, a compound of formula (I) is used at a rate of 0.0001 g to 10 g (for example 0.001 g or 0.05 g), preferably 0.005 g to 10 g, more preferably 0.005 g to 4 g, per kilogram of seed.

In another aspect the present invention provides a composition for crop enhancement comprising a crop enhancing amount of a compound of formula (I) and a suitable carrier or diluent therefor.

In a still further aspect the invention provides a method of crop enhancement which comprises treating the pests or the locus of the pests with a crop enhancing amount of a composition comprising a compound of formula (I).

The compositions can be chosen from a number of formulation types, including dustable powders (DP), soluble powders (SP), water soluble granules (SG), water dispersible granules (WG), wettable powders (WP), granules (GR) (slow or fast release), soluble concentrates (SL), oil miscible liquids (OL), ultra low volume liquids (UL), emulsifiable concentrates (EC), dispersible concentrates (DC), emulsions (both oil in water (EW) and water in oil (EO)), micro-emulsions (ME), suspension concentrates (SC), oil-based suspension concentrate (OD), aerosols, fogging/smoke formulations, capsule suspensions (CS) and seed treatment formulations. The formulation type chosen in any instance will depend upon the particular purpose en-visaged and the physical, chemical and biological properties of the compound of formula (I).

Dustable powders (DP) may be prepared by mixing a compound of formula (I) with one or more solid diluents (for example natural clays, kaolin, pyrophyllite, bentonite, alumina, montmorillonite, kieselguhr, chalk, diatomaceous earths, calcium phosphates, calcium and magnesium carbonates, sulphur, lime, flours, talc and other organic and inorganic solid carriers) and mechanically grinding the mixture to a fine powder.

Soluble powders (SP) may be prepared by mixing a compound of formula (I) with one or more water-soluble inorganic salts (such as sodium bicarbonate, sodium carbonate or magnesium sulphate) or one or more water-soluble organic solids (such as a polysaccharide) and, optionally, one or more wetting agents, one or more dispersing agents or a mixture of said agents to improve water dispersibility/solubility. The mixture is then ground to a fine powder. Similar compositions may also be granulated to form water soluble granules (SG).

Wettable powders (WP) may be prepared by mixing a compound of formula (I) with one or more solid diluents or carriers, one or more wetting agents and, preferably, one or more dispersing agents and, optionally, one or more suspending agents to facilitate the dispersion in liquids. The mixture is then ground to a fine powder. Similar compositions may also be granulated to form water dispersible granules (WG).

Granules (GR) may be formed either by granulating a mixture of a compound of formula (I) and one or more powdered solid diluents or carriers, or from pre-formed blank granules by absorbing a compound of formula (I) (or a solution thereof, in a suitable agent) in a porous granular material (such as pumice, attapulgite clays, fuller's earth, kieselguhr, diatomaceous earths or ground corn cobs) or by adsorbing a compound of formula (I) (or a solution thereof, in a suitable agent) on to a hard core material (such as sands, silicates, mineral carbonates, sulphates or phosphates) and drying if necessary. Agents which are commonly used to aid absorption or adsorption include solvents (such as aliphatic and aromatic petroleum solvents, alcohols, ethers, ketones and esters) and sticking agents (such as polyvinyl acetates, polyvinyl alcohols, dextrins, sugars and vegetable oils). One or more other additives may also be included in granules (for example an emulsifying agent, wetting agent or dispersing agent).

Dispersible Concentrates (DC) may be prepared by dissolving a compound of formula (I) in water or an organic solvent, such as a ketone, alcohol or glycol ether. These solutions may contain a surface active agent (for example to improve water dilution or prevent crystallisation in a spray tank).

Emulsifiable concentrates (EC) or oil-in-water emulsions (EW) may be prepared by dissolving a compound of formula (I) in an organic solvent (optionally containing one or more wetting agents, one or more emulsifying agents or a mixture of said agents). Suitable organic solvents for use in ECs include aromatic hydrocarbons (such as alkylbenzenes or alkylnaphthalenes, exemplified by SOLVESSO 100, SOLVESSO 150 and SOLVESSO 200; SOLVESSO is a Registered Trade Mark), ketones (such as cyclohexanone or methylcyclohexanone) and alcohols (such as benzyl alcohol, furfuryl alcohol or butanol), N-alkylpyrrolidones (such as N-methylpyrrolidone or N-octylpyrrolidone), dimethyl amides of fatty acids (such as C₈-C₁₀ fatty acid dimethylamide) and chlorinated hydrocarbons. An EC product may spontaneously emulsify on addition to water, to produce an emulsion with sufficient stability to allow spray application through appropriate equipment. Preparation of an EW involves obtaining a compound of formula (I) either as a liquid (if it is not a liquid at room temperature, it may be melted at a reasonable temperature, typically below 70° C.) or in solution (by dissolving it in an appropriate solvent) and then emulsifiying the resultant liquid or solution into water containing one or more SFAs, under high shear, to produce an emulsion. Suitable solvents for use in EWs include vegetable oils, chlorinated hydrocarbons (such as chlorobenzenes), aromatic solvents (such as alkylbenzenes or alkylnaphthalenes) and other appropriate organic solvents which have a low solubility in water.

Microemulsions (ME) may be prepared by mixing water with a blend of one or more solvents with one or more SFAs, to produce spontaneously a thermodynamically stable isotropic liquid formulation. A compound of formula (I) is present initially in either the water or the solvent/SFA blend. Suitable solvents for use in MEs include those hereinbefore described for use in ECs or in EWs. An ME may be either an oil-in-water or a water-in-oil system (which system is present may be determined by conductivity measurements) and may be suitable for mixing water-soluble and oil-soluble pesticides in the same formulation. An ME is suitable for dilution into water, either remaining as a microemulsion or forming a conventional oil-in-water emulsion.

Suspension concentrates (SC) may comprise aqueous or non-aqueous suspensions of finely divided insoluble solid particles of a compound of formula (I). SCs may be prepared by ball or bead milling the solid compound of formula (I) in a suitable medium, optionally with one or more dispersing agents, to produce a fine particle suspension of the compound. One or more wetting agents may be included in the composition and a suspending agent may be included to reduce the rate at which the particles settle. Alternatively, a compound of formula (I) may be dry milled and added to water, containing agents hereinbefore described, to produce the desired end product.

Oil-based suspension concentrate (OD) may be prepared similarly by suspending finely divided insoluble solid particles of a compound of formula (I) in an organic fluid (for example at least one mineral oil or vegetable oil). ODs may further comprise at least one penetration promoter (for example an alcohol ethoxylate or a related compound), at least one non-ionic surfactants and/or at least one anionic surfactant, and optionally at least one additive from the group of emulsifiers, foam-inhibiting agents, preservatives, anti-oxidants, dyestuffs, and/or inert filler materials. An OD is intended and suitable for dilution with water before use to produce a spray solution with sufficient stability to allow spray application through appropriate equipment.

Aerosol formulations comprise a compound of formula (I) and a suitable propellant (for example n-butane). A compound of formula (I) may also be dissolved or dispersed in a suitable medium (for example water or a water miscible liquid, such as n-propanol) to provide compositions for use in non-pressurised, hand-actuated spray pumps.

A compound of formula (I) may be mixed in the dry state with a pyrotechnic mixture to form a composition suitable for generating, in an enclosed space, a smoke containing the compound.

Capsule suspensions (CS) may be prepared in a manner similar to the preparation of EW formulations but with an additional polymerisation stage such that an aqueous dispersion of oil droplets is obtained, in which each oil droplet is encapsulated by a polymeric shell and contains a compound of formula (I) and, optionally, a carrier or diluent therefor. The polymeric shell may be produced by either an interfacial polycondensation reaction or by a coacervation procedure. The compositions may provide for controlled release of the compound of formula (I) and they may be used for seed treatment. A compound of formula (I) may also be formulated in a biodegradable polymeric matrix to provide a slow, controlled release of the compound.

A compound of formula (I) may also be formulated for use as a seed treatment, for example as a powder composition, including a powder for dry seed treatment (DS), a water soluble powder (SS) or a water dispersible powder for slurry treatment (WS), or as a liquid composition, including a flowable concentrate (FS), a solution (LS) or a capsule suspension (CS). The preparations of DS, SS, WS, FS and LS compositions are very similar to those of, respectively, DP, SP, WP, SC, OD and DC compositions described above. Compositions for treating seed may include an agent for assisting the adhesion of the composition to the seed (for example a mineral oil or a film-forming barrier).

A composition used according to the present invention may include one or more additives to improve the biological performance of the composition (for example by improving wetting, retention or distribution on surfaces; resistance to rain on treated surfaces; or uptake or mobility of a compound of formula (I)). Such additives include surface active agents (SFAs), spray additives based on oils, for example certain mineral oils, vegetable oils or natural plant oils (such as soy bean and rape seed oil), and blends of these with other bio-enhancing adjuvants (ingredients which may aid or modify the action of a compound of formula (I)). Increasing the effect of a compound of formula (I) may for example be achieved by adding ammonium and/or phosphonium salts, and/or optionally at least one penetration promoter such as fatty alcohol alkoxylates (for example rape oil methyl ester) or vegetable oil esters.

Wetting agents, dispersing agents and emulsifying agents may be surface active agents (SFAs) of the cationic, anionic, amphoteric or non-ionic type.

Suitable SFAs of the cationic type include quaternary ammonium compounds (for example cetyltrimethyl ammonium bromide), imidazolines and amine salts.

Suitable anionic SFAs include alkali metals salts of fatty acids, salts of aliphatic monoesters of sulphuric acid (for example sodium lauryl sulphate), salts of sulphonated aromatic compounds (for example sodium dodecylbenzenesulphonate, calcium dodecylbenzenesulphonate, butylnaphthalene sulphonate and mixtures of sodium di-isopropyl- and tri-isopropyl-naphthalene sulphonates), ether sulphates, alcohol ether sulphates (for example sodium laureth-3-sulphate), ether carboxylates (for example sodium laureth-3-carboxylate), phosphate esters (products from the reaction between one or more fatty alcohols and phosphoric acid (predominately mono-esters) or phosphorus pentoxide (predominately di-esters), for example the reaction between lauryl alcohol and tetraphosphoric acid; additionally these products may be ethoxylated), sulphosuccinamates, paraffin or olefine sulphonates, taurates and lignosulphonates.

Suitable SFAs of the amphoteric type include betaines, propionates and glycinates.

Suitable SFAs of the non-ionic type include condensation products of alkylene oxides, such as ethylene oxide, propylene oxide, butylene oxide or mixtures thereof, with fatty alcohols (such as oleyl alcohol or cetyl alcohol) or with alkylphenols (such as octylphenol, nonylphenol or octylcresol); partial esters derived from long chain fatty acids or hexitol anhydrides; condensation products of said partial esters with ethylene oxide; block polymers (comprising ethylene oxide and propylene oxide); alkanolamides; simple esters (for example fatty acid polyethylene glycol esters); amine oxides (for example lauryl dimethyl amine oxide); and lecithins.

Suitable suspending agents include hydrophilic colloids (such as polysaccharides, polyvinylpyrrolidone or sodium carboxymethylcellulose) and swelling clays (such as bentonite or attapulgite).

A compound of formula (I) may be applied by any of the known means of applying agricultural compositions. For example, it may be applied, formulated or unformulated, to the locus of the crops, directly to the crops, including to any part of the plant, including the foliage, stems, branches or roots, to the seed before it is planted, or to the media in which plants are growing or are to be planted (such as soil surrounding the roots, the soil generally, paddy water or hydroponic culture systems), directly or it may be sprayed on, dusted on, applied by dipping, applied as a cream or paste formulation, applied as a vapour or applied through distribution or incorporation of a composition (such as a granular composition or a composition packed in a water-soluble bag) in soil or an aqueous environment.

A compound of formula (I) may also be injected into plants or sprayed onto vegetation using electrodynamic spraying techniques or other low volume methods, or applied by land or aerial irrigation systems.

Compositions for use as aqueous preparations (aqueous solutions or dispersions) are generally supplied in the form of a concentrate containing a high proportion of the active ingredient, the concentrate being added to water before use. These concentrates, which may include DCs, SCs, ODs, ECs, EWs, MEs SGs, SPs, WPs, WGs and CSs, are often required to withstand storage for prolonged periods and, after such storage, to be capable of addition to water to form aqueous preparations which remain homogeneous for a sufficient time to enable them to be applied by conventional spray equipment. Such aqueous preparations may contain varying amounts of a compound of formula (I) (for example 0.0001 to 10%, by weight) depending upon the purpose for which they are to be used.

A compound of formula (I) may be used in mixtures with fertilisers (for example nitrogen-, potassium- or phosphorus-containing fertilisers, and more particularly ammonium nitrate and/or urea fertilizers). Suitable formulation types include granules of fertiliser. The mixtures suitably contain up to 25% by weight of the compound of formula (I).

The invention therefore also provides a fertiliser composition comprising a fertiliser and a compound of formula (I).

In a further aspect of the present invention, the compounds or composition of the present invention may be applied in combination with one or more compounds having a pesticidal effect. Such compounds include those that possess fungicidal, herbicidal, safening, insecticidal, nematicidal or acaricidal activity.

In a further aspect of the present invention, the compounds or composition of the present invention may be applied in combination with one or more other compounds having a crop enhancement effect. Such compounds include micronutrients, saccharides, amino acids, flavonoids, quinines, and plant activators/growth stimulators. For example, such compounds include natural or synthetic hormones, auxins, brassinosteroids, gibberellins, abscisic acid, cytokinins, jasmonates, strigolactones, salicylic acid, ethylene, 1-methylcyclopropene, trinexapac-ethyl or derivatives thereof. Such compounds also include pesticides that have a crop enhancement effect, for example strobilurins (including azoxystrobin, pyraclostrobin), and neonicotinoids (including thiamethoxam, and imidacloprid).

Examples of compounds of formula (I) are given in Tables A and B below.

TABLE A
Compounds of formula (I), more polar
diastereoisomer (R2 = R3 = R4 = R5 =, R6 = Me, W = O)

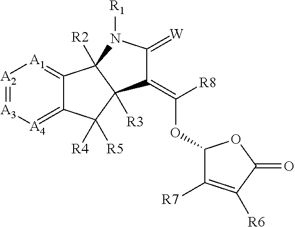

| Compound | R1 | A₁ | A₂ | A₃ | A₄ | R7 |
|---|---|---|---|---|---|---|
| A1 | H | C—H | C—H | C—H | C—H | H |
| A2 | H | C—H | C—H | C—H | C—Br | H |
| A3 | Ph | C—H | C—H | C—H | C—H | H |
| A4 | CH₃C(O) | C—H | C—H | C—H | C—H | H |
| A5* | tBuOC(O) | C—H | C—H | C—H | C—H | H |
| A6* | H | C—CN | C—H | C—H | C—H | H |
| A7 | H | C—H | C—H | C—H | C—H | Me |
| A8 | Me | C—H | C—H | C—H | C—H | H |
| A9* | H | C—H | C—H | C—H | C—Me | H |

*1/1 mixture of diastereoisomers.

TABLE B
Compounds of formula (I), more polar diastereoisomer
(R2 = R3 = R4 = R5 = R7 = H, R6 = Me, W = O)

| | |
|---|---|
| | (I) |
| 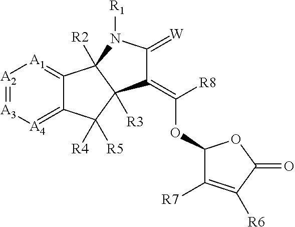 | |

| Compound | R1 | A₁ | A₂ | A₃ | A₄ |
|---|---|---|---|---|---|
| B1 | H | C—H | C—H | C—H | C—H |

### BIOLOGICAL EXAMPLES

### Example B1

### Enhancement of Soybean Seedling Emergence in Saturated Cold Test

Soybean seeds cv. Toliman were treated with a test compound of formula I formulated as FS100, at a dose of 0.05 g ai/kg, 0.005 g ai/kg, or 0.0001 g ai/kg seeds. Seeds were planted on 2 cm levelled pre-chilled moist sand and covered with a 2 cm layer of a mixture of steamed soil and sand (1:1). Enough cold water (10° C.) was added to the sand-soil mixture to adjust soil moisture to 60% water holding capacity. The seeds were incubated at 10° C. in the dark for 10 days and transferred into a chamber with alternating temperature (25° C. day/23° C. night) and light source (12 h) for 10 days. Seedling emergence in % and dry mass of above parts of seedling per tray was determined at the end of the incubation period. The test was performed using four replicates (4×50 seeds).

TABLE 1
Effect of compounds of formula I on seedling emergence
and total shoot mass of soybeans in saturated cold test

| Treatment | Dose | Seedling | Total shoot |
|---|---|---|---|
| (compound | (g ai/ | emergence | dry mass |
| number) | kg seeds) | (%) | (g) |
| Untreated | n/a | 57.5 | 4.515 |
| control | | | |
| A3 | 0.05 | 51.5 | 3.850 |
| | 0.005 | 57 | 4.540 |
| | 0.0001 | 67 | 4.838 |
| A4 | 0.05 | 61.5 | 4.850 |
| | 0.005 | 62.5 | 4.550 |
| | 0.0001 | 62 | 4.788 |
| A5 | 0.05 | 55 | 4.105 |
| | 0.005 | 51.5 | 3.815 |
| | 0.0001 | 48 | 3.420 |
| A2 | 0.05 | 56 | 3.993 |
| | 0.005 | 49.5 | 3.725 |
| | 0.0001 | 48.5 | 3.810 |
| B1 | 0.05 | 44.5 | 3.365 |
| | 0.005 | 67 | 5.065 |
| | 0.0001 | 58.5 | 4.440 |
| A1 | 0.05 | 54.5 | 4.438 |
| | 0.005 | 63.5 | 5.018 |
| | 0.0001 | 67.5 | 5.298 |

The results show that compounds of formula I generally enhance seedling emergence, and/or increase shoot dry mass of soybeans grown under cold stress conditions.

### Example B2

### Enhancement of Soybean Seedling Emergence in Saturated Warm Test

Treated seeds used in the saturated cold test were evaluated in parallel in a saturated warm test. Test conditions were the same as those described for the 10 day warm incubation period of the saturated cold test. Briefly, seeds were placed onto a saturated sand layer of 2 cm and covered with saturated sand-soil mixture (60% WHC) at room temperature. The seeds were incubated 10 days at 25° C. day/23° C. night and 12 h light. Seedling emergence and shoot dry mass was determined as described in the saturated cold test method. The test was performed using four replicates each 50 seeds. The results are shown in Table 2.

TABLE 2
Effect of compounds of formula I on seedling emergence
and total shoot mass of soybeans in saturated warm test

| Treatment | Dose | Seedling | Total shoot |
|---|---|---|---|
| (compound | (g ai/ | emergence | dry mass |
| number) | kg seeds) | (%) | (g) |
| Untreated | n/a | 89.5 | 5.815 |
| control | | | |
| A3 | 0.05 | 81 | 5.918 |
| | 0.005 | 87 | 6.235 |
| | 0.0001 | 91 | 6.158 |
| A4 | 0.05 | 86 | 5.920 |
| | 0.005 | 90 | 5.908 |
| | 0.0001 | 89 | 6.035 |
| A5 | 0.05 | 91.5 | 6.148 |
| | 0.005 | 91.5 | 6.095 |
| | 0.0001 | 91 | 6.260 |
| A2 | 0.05 | 91.5 | 6.325 |
| | 0.005 | 87.5 | 6.105 |
| | 0.0001 | 89 | 5.763 |
| B1 | 0.05 | 89 | 5.935 |
| | 0.005 | 94 | 6.440 |
| | 0.0001 | 91 | 5.778 |
| A1 | 0.05 | 91 | 6.023 |
| | 0.005 | 89 | 6.010 |
| | 0.0001 | 88.5 | 6.053 |

The results show that compounds of formula I generally enhance seedling emergence and/or increase shoot dry mass of soybeans grown under warm conditions in wet soil.

### Example B3

### Enhancement of Germination of Brassica oleracea cv Botrytis

The effect of compounds of Formula (I) on the germination of Brassica oleracea cv Botrytis or common cauliflower was tested on two types of cauliflowers: temperate types and tropical types. These two types were chosen because they display different sensitivities to the light conditions and temperature during germination. Germination of a sensitive temperate type is inhibited by light at 10° C. while for the tropical types germination at 20° is stimulated by the presence of light. Hence, 10° C. in the light and 20° C. in the dark are considered suboptimal or stress conditions for germination of the two types, respectively.

The temperate seed batches tested are part of commercially produced seed batches of various varieties which are known to be sensitive to light at 10° C. These seeds were harvested and cleaned according standard commercial procedures. Ready seed batches were used (Ready indicates the processing level of these seeds: they have been cleaned and sized but received no other treatments). The tropical seed batches tested are part of seed batches produced as basic seed (for maintenance of the parental line) and were processed accordingly.

Germination was assessed using the standard paper germination test for Brassica: Fifty seeds were placed on blue germination paper, which was moistened with the appropriate solutions, in closed oblong germination boxes. Each condition was tested in duplo. Germination boxes were placed in controlled germination cabinets with the appropriate temperature and light conditions. Germination of seeds was counted at regular intervals. Seeds were considered to be germinated when the radical had protruded the testa and endosperm (radical size approximately 1 mm).

Test compounds were dissolved in DMSO at a concentration of 50 mM and stored at −20° C. The strigolactone analogue GR24 (commercially available as a racemic mixture of 2 diastereoisomers, referred to as “synthetic strigolactone GR-24” and first prepared by Johnson A. W. & all, Journal of the Chemical Society, Perkin Transactions 1, 1981, page 1734-1743) was included as positive control. Germination solutions were prepared by diluting the stock solutions with demineralized water till 25 μM. As control solutions demineralized water and a 0.05% v/v DMSO solution were used.

The effect of the strigolactone derivatives on germination is shown in table 3. These results show that strigolactones stimulate germination at suboptimal conditions.

TABLE 3
Germination of seeds of the temperate cauliflower Spacestar (seed batch
11B313; produced in South Africa in 2010 (cold-sensitive) and seed
batch 11B314; produced in Chile in 2010 (not cold sensitive)) in the
presence of 25 μM of the different strigolactone derivatives
at 10° C. and in the light A: First set of Strigolactones;
B: Second set of Strigolactones. Sets were tested in seperate
experiments and with two independent experiments for each set.

| | Spacestar 11B313 | | Spacestar 11B314 | |
|---|---|---|---|---|
| | Gₘₐₓ^{a} | stimulation^{b} | Gₘₐₓ^{a} | stimulation^{b} |
| Compound | (%) | (%) | (%) | (%) |

A

| | | | | |
|---|---|---|---|---|
| DMSO | 60.0 | 0.0 | 90.5 | 0.0 |
| GR24 | 71.5 | 19.2 | 93.5 | 3.3 |
| A1 | 77.0 | 28.3 | 90.5 | 0.0 |
| A5 | 74.5 | 24.2 | 92.5 | 2.2 |
| A4 | 82.0 | 36.7 | 91.0 | 0.6 |
| A3 | 74.5 | 24.2 | 89.5 | −1.1 |
| B1 | 60.5 | 0.8 | 93.5 | 3.3 |

B

| | | | | |
|---|---|---|---|---|
| DMSO | 54.0 | 0.0 | 86.0 | 0.0 |
| GR24 | 80.0 | 48.1 | 93.0 | 8.1 |
| A7 | 68.5 | 26.9 | 95.0 | 10.5 |
| A8 | 73.0 | 35.2 | 93.5 | 8.7 |
| A9 | 59.8 | 10.6 | 94.0 | 9.3 |

^{a}total germination as percentage of sown seeds
^{b}extra germination compared to the DMSO treatment (control), expressed as percentage of the germination in the DMSO treatment

1. A method of enhancing crop plants by applying to the plants, plant parts, plant propagation material, or a plant growing locus, a compound of formula (I): 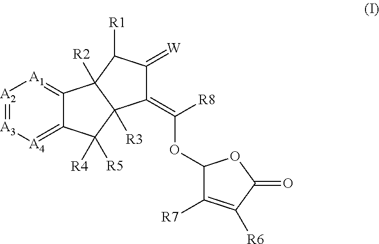
wherein
W is O or S;
R2 and R3 are independently hydrogen, or C1-C3 alkyl;
R4 and R5 are independently hydrogen, halogen, nitro, cyano, C1-C3 alkyl, C1-C3 haloalkyl, C1-C3 alkoxy, hydroxyl, —OC(O)R9, amine, N— C1-C3 alkyl amine, or N,N-di-C1-C3 alkyl amine;
R9 is hydrogen, C1-C6 alkyl, C1-C6 alkoxy, or C1-C6 haloalkyl;
R6 and R7 are independently hydrogen, C1-C3 alkyl, hydroxyl, or C1-C3 alkoxy;
R8 is hydrogen, nitro, cyano, C1-C6 alkyl, or C1-C6 haloalkyl;
R1 is hydrogen, C1-C6 alkoxy, hydroxyl, amine, N— C1-C6 alkyl amine, N,N-di-C1-C6 alkyl amine, C1-C6 alkyl optionally substituted by one to five R10, C1-C8 alkylcarbonyl, C1-C8 alkoxycarbonyl, aryl optionally substituted by one to five R10, heteroaryl optionally substituted by one to five R10, heterocyclyl optionally substituted by one to five R10, or benzyl optionally substituted by one to five R10;
R10 is hydrogen, cyano, nitro, halogen, C1-C6 alkyl, C1-C6 alkoxy, C1-C6 haloalkyl, C2-C6 alkenyl, or C2-C6 alkynyl;
A₁, A₂, A₃ and A₄ are each independently C—X or nitrogen, wherein each X may be the same or different, and provided that no more than two of A₁, A₂, A₃ and A₄ are nitrogen;
and X is hydrogen, halogen, cyano, hydroxyl, —OC(O)R9, C1-C6 alkoxy, C1-C6 alkyl, C1-C6 haloalkyl, C1-C3 hydroxyalkyl, nitro, amine, N— C1-C6 alkyl amine, N,N-di-C1-C6 alkyl amine, or NHC(O)R9;
or a composition comprising said compound.

2. A method according to claim 1 for improving plant yield, comprising applying to a plant, plant part, plant propagation material, or a plant growing locus, a compound of formula (I).

3. A method according to claim 1 for improving plant input use efficiency, comprising applying to a plant, plant part, plant propagation material, or a plant growing locus, a compound of formula (I).

4. A method according to claim 1 for improving plant vigour and/or plant quality, and/or plant tolerance to stress factors, comprising applying to a plant, plant part, plant propagation material, or a plant growing locus, a compound of formula (I).

5. A method according to claim 1, wherein in the compound of formula (I):
W is Oxygen;
R2 and R3 are independently hydrogen, methyl or ethyl;
R4 and R5 are independently hydrogen, hydroxyl, methyl or ethyl;
R6, R7 and R8 are independently hydrogen, methyl or ethyl;
R1 is hydrogen, C1-C6 alkoxy, C1-C6 alkyl optionally substituted by one to five R10, C1-C8 alkylcarbonyl, C1-C8 alkoxycarbonyl, aryl optionally substituted by one to five R10, heteroaryl optionally substituted by one to five R10, heterocyclyl optionally substituted by one to five R10, or benzyl optionally substituted by one to five R10;
R10 is independently hydrogen, cyano, nitro, halogen, C1-C6 alkyl, C1-C6 alkoxy or C1-C6 haloalkyl;
A₁, A₂, A₃ and A₄ are each independently C-X; and
X is hydrogen, hydroxyl, halogen, cyano, methyl, ethyl, n-propyl, hydroxymethyl, trifluoromethyl or methoxy.

6. A method according to claim 1, wherein in the compound of formula (I):
W is oxygen;
R2, R3, R7 and R8 are hydrogen;
R4 and R5 are independently hydrogen or hydroxyl;
R6 is methyl;
R1 is hydrogen, C1-C6 alkoxy, C1-C6 alkyl optionally substituted by one to five R10, C1-C8 alkylcarbonyl, C1-C8 alkoxycarbonyl, benzyl optionally substituted by one to five R10;
R10 is hydrogen, cyano, nitro, chloride, bromine, fluorine, methyl, methoxy and trifluoromethyl;
A₁, A₂, A₃ and A₄ are C-X; and
X is hydrogen, hydroxyl, methyl, trifluoromethyl or methoxy.

7. A method according to claim 1, wherein the compound of formula (I) is applied in the form of a composition, further comprising an agriculturally acceptable carrier.

8. (canceled)
